(19) United States
(12) Patent Application Publication
(10) Pub. No.: US 2024/0410893 A1
(43) Pub. Date: Dec. 12, 2024

(54) COMPOSITIONS, SYSTEMS, AND METHODS FOR DETERMINATION OF T2R AND/OR T1R PHENOTYPE IN INDIVIDUALS TO PREDICT CANNABIS TASTING PREFERENCES, CHARACTERIZATION OF CANNABIS IN RELATION TO INDIVIDUAL TASTING PREFERENCES, AND SELECTION OF CANNABIS

(71) Applicant: Phenomune, LLC, The Woodlands, TX (US)

(72) Inventor: Henry P Barham, Baton Rouge, LA (US)

(73) Assignee: Phenomune, LLC, The Woodlands, TX (US)

(21) Appl. No.: 18/454,598

(22) Filed: Aug. 23, 2023

Related U.S. Application Data
(63) Continuation-in-part of application No. 17/693,928, filed on Mar. 14, 2022.
(63) Continuation-in-part of application No. 17/500,755, filed on Oct. 13, 2021, now abandoned.
(60) Provisional application No. 63/400,215, filed on Aug. 23, 2022.
(60) Provisional application No. 63/391,246, filed on Jul. 21, 2022.
(60) Provisional application No. 63/356,739, filed on Jun. 29, 2022.
(60) Provisional application No. 63/353,571, filed on Jun. 18, 2022.
(60) Provisional application No. 63/350,397, filed on Jun. 8, 2022.
(60) Provisional application No. 63/340,630, filed on May 11, 2022.
(60) Provisional application No. 63/092,279, filed on Oct. 15, 2020.

(51) Int. Cl. G01N 33/68

(52) U.S. Cl. CPC G01N 33/68; G01N 2333/705

(57) ABSTRACT

A method of measuring phenotypic expression of T2Rs and/or T1Rs for a subject to predict taste preferences for cannabis may include stimulating T2Rs and/or T1Rs of a subject with agonists, and detecting products released as a result of stimulation of the T2Rs and/or T1Rs. A method may include recording discerned level of taste perception after stimulation with agonists and correlating the discerned level to phenotypic expression, which may be used to predict taste preferences for cannabis profiles. A method may include calculating cannabis bin scores for predicted cannabis preferences, from phenotypic expression of T2Rs and/or T1Rs, obtaining taste preference information, and determining the subject's taste preference scores for cannabis administered to the subject.

### CROSS-REFERENCE TO RELATED APPLICATIONS

This application is related and claims priority to each of the following: U.S. Provisional Application No. 63/400,215, filed Aug. 23, 2022, titled T1R PHENOTYPE IN INDIVIDUALS TO PREDICT CANNABIS TASTING PREFERENCES, CHARACTERIZATION OF CANNABIS IN RELATION TO INDIVIDUAL TASTING PREFERENCES, AND SELECTION OF CANNABIS; U.S. Provisional Application No. 63/391,246, filed Jul. 21, 2022, titled COMPOSITIONS, SYSTEMS, AND METHODS FOR PREDICTING GENOTYPE FROM PHENOTYPIC EXPRESSION OF T2RS AND TEST METHODS THEREOF, which is incorporated herein by reference in entirety; U.S. Provisional Application No. 63/356,739, filed Jun. 29, 2022 titled CHARACTERIZATION OF PHENOTYPIC EXPRESSION OF BITTER TASTE RECEPTORS FOR THE GUT MICROBIOME AND APPLICATIONS THEREOF, which is incorporated herein by reference in entirety; U.S. Provisional Application No. 63/353,571, filed Jun. 18, 2022 titled COMPOSITIONS, SYSTEMS, AND METHODS FOR TREATMENT WITH T2R AGONISTS FROM TERPENES, PLANT MATERIAL, CANNABIS PLANT MATERIAL, AND HEMP PLANT MATERIAL, which is incorporated herein by reference in entirety; U.S. Provisional Application No. 63/350,397, filed Jun. 8, 2022 titled COMPOSITIONS, SYSTEMS, AND METHODS FOR DETERMINATION OF T2R AND/OR T1R PHENOTYPE IN INDIVIDUALS TO PREDICT WINE TASTING PREFERENCES, CHARACTERIZATION OF WINE IN RELATION TO INDIVIDUAL TASTING PREFERENCES, AND SELECTION OF WINE, which is incorporated herein by reference in entirety; U.S. Provisional Application No. 63/340,630, filed May 11, 2022 titled COMPOSITIONS, SYSTEMS, AND METHODS FOR TREATMENT WITH T2R AGONIST NASAL SPRAYS AND APPLICATIONS THEREOF, which is incorporated herein by reference in entirety; U.S. Non-provisional application Ser. No. 17/693,928, filed Mar. 14, 2022, titled TESTING METHODS FOR DETERMINATION OF T2R PHENOTYPE AND APPLICATIONS THEREOF, which is incorporated herein by reference in entirety; U.S. Non-provisional application Ser. No. 17/500,755, filed Oct. 13, 2021, titled TESTING METHODS FOR DETERMINATION OF T2R PHENOTYPE AND APPLICATIONS THEREOF, which is incorporated herein by reference in entirety; and U.S. Provisional Application No. 63/092,279, filed Oct. 15, 2020, titled TESTING METHODS FOR DETERMINATION OF T2R PHENOTYPE AND APPLICATIONS THEREOF, which is incorporated herein by reference in entirety.

### FIELD OF THE INVENTION20

The disclosure relates to the characterization of chemosensory receptors, stimulation of chemosensory receptors, and applications of stimulating chemosensory receptors. More particularly, the disclosure relates to characterization and stimulation of T2Rs and/or T1Rs and applications of stimulating T2Rs and/or T1Rs. The disclosure relates to compositions, systems and methods for determination of T2R and/or T1R phenotype expression in individuals to predict or characterize individual cannabis tasting preferences, characterization of cannabis in relation to individual taste preferences arising from T2R and/or T1R phenotype expression in individuals, and selection of cannabis in relation to predicted or characterized individual cannabis tasting preferences arising from T2R and/or T1R phenotype expression in individuals, which may be determined by applications of compositions, systems and methods as herein disclosed.

### BACKGROUND OF THE INVENTION

Chemosensory receptors may be encoded by six families of genes including trace amine-associated receptors (TAAR), olfactory receptors (OR), vomeronasal receptor type 1 and 2 (V1R and V2R), and taste receptors type 1 and 2 (T1R and T2R). All of the chemosensory receptor proteins may be G-protein coupled receptors. Chemosensory receptors may be expressed on the surface of solitary chemosensory cells (SCCs). Receptors belonging to Taste Receptor Family-1 subtypes 2 and 3 (T1R2/T1R3) detect sweet compounds such as glucose and sucrose. Taste Receptor Family-2 receptors (T2Rs) detect bitter taste compounds. Greater than fifty (50) T2Rs have been characterized. Stimulation of T2Rs activates, at least, the canonical taste signaling cascade involving phospholipase C β2 (PLCβ2) and transient receptor potential cation channel subfamily M member 5 [Nei, M., Niimura, Y. & Nozawa, M. The evolution of animal chemosensory receptor gene repertoires: roles of chance and necessity. Nature Reviews Genetics 9, 951-963 (2008)]. The aforementioned manuscript is hereby incorporated by reference in entirety. Unless expressly stated to the contrary, the form of instantiation of any element of this disclosure shall be considered as being only a matter of design choice.

T2Rs may be genetically diverse, a phenomenon that helps to explain the wide variety of taste preferences both within and between cultures. Many individuals find various bitter foods to be detestable, while others do not have the same aversive response. This genetic variation of T2Rs may be found on the tongue, but not exclusively. More recently, bitter and sweet receptors have been discovered in a variety of extra-oral tissues including the brain, thyroid, pancreas, testes and throughout the respiratory and gastrointestinal tracts. The activity of T2Rs and/or T1Rs may be useful in predicting tast preferences. There is need for improved compositions, systems and methods for determination of T2R and/or T1R phenotype expression in individuals to predict or characterize individual cannabis tasting preferences. There is need for improved compositions, systems and methods for determination of T2R and/or T1R phenotype expression in individuals to characterize cannabis in relation to individual taste preferences arising from T2R and/or T1R phenotype expression in individuals. There is need for characterization and selection of cannabis in relation to predicted or characterized individual cannabis tasting preferences arising from T2R and/or T1R phenotype expression in individuals, which may be determined by applications of improved compositions, systems and methods as herein disclosed.

The above-mentioned shortcomings, disadvantages and problems may be addressed herein, as will be understood by those skilled in the art upon reading and studying the following specification. This summary is provided to introduce a selection of concepts in simplified form that may be further described below in more detail in the Detailed Description. This summary is not intended to identify key or essential features of the claimed subject matter. Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

### BRIEF SUMMARY OF THE INVENTION

Need exists for reliable methods of predicting tast preferences of cannabis. Phenotypic expression of T2Rs and/or T1Rs may correlated to taste preferences of different types of cannabis.

In embodiments, a method for predicting cannabis preferences of a subject may be provided, the method comprising: first determining phenotypic expression of T2Rs and/or T1Rs, wherein the determining may be from an actual test administered to detect the functionality of T2Rs and/or T1Rs; obtaining taste preference information from the subject; second determining the subject's taste preference scores for one or more cannabis administered to the subject; predicting cannabis preferences for the subject.

In embodiments, a method for predicting cannabis preferences of a subject may be provided, the method comprising: first determining phenotypic expression of T2Rs and/or T1Rs, wherein the determining may be from an actual test administered to detect the functionality of T2Rs and/or T1Rs; second determining the subject's taste preference scores for one or more cannabis administered to the subject; predicting cannabis preferences for the subject.

In embodiments, a method for predicting cannabis preferences of a subject may be provided, the method comprising: first determining phenotypic expression of T2Rs and/or T1Rs, wherein the determining may be from an actual test administered to detect the functionality of T2Rs and/or T1Rs; predicting cannabis preferences for the subject.

Apparatus, systems, and methods of varying scope may be described herein. These aspects may be indicative of various non-limiting ways in which the disclosed subject matter may be utilized, all of which may be intended to be within the scope of the disclosed subject matter. In addition to the aspects and advantages described in this summary, further aspects, features, and advantages will become apparent by reference to the associated drawings, detailed description, and claims.

### BRIEF DESCRIPTION OF THE DRAWINGS

The novel features believed characteristic of the disclosed subject matter will be set forth in any claims that are filed later. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 is a simplified flow diagram illustrating aspects of a method 100 of treatment in an embodiment;

FIG. 2 is a simplified flow diagram illustrating aspects of a method 200 of treatment in an embodiment;

FIG. 3 is a simplified flow diagram illustrating aspects of a method 300 of treatment in an embodiment.

### DETAILED DESCRIPTION OF ILLUSTRATIVE AN EMBODIMENT

In this detailed description, reference is made to the accompanying drawings which form a part hereof, and in which is shown by way of illustration specific an embodiment which may be practiced. Reference now should be made to the drawings, in which the same reference numbers are used throughout the different figures to designate the same components. These an embodiment are described in sufficient detail to enable those skilled in the art to practice the an embodiment and disclosure. It is to be understood that other an embodiment may be utilized, and that logical, mechanical, electrical, and other changes may be made without departing from the scope of the an embodiment and disclosure. In view of the foregoing, the following detailed description is not to be taken as limiting the scope of the an embodiment or disclosure.

The terminology used herein is for the purpose of describing particular an embodiment only and is not intended to be limiting. As used herein, the singular forms “a”, “an”, and “the” are intended to include the plural forms as well, unless the context clearly indicates otherwise. It will be further understood that the terms “comprises” and/or “comprising” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be appreciated that for simplicity and clarity of illustration, where considered appropriate, reference numerals may be repeated among the figures to indicate corresponding or analogous elements. In addition, numerous specific details are set forth in order to provide a thorough understanding of the implementations described herein. However, it will be understood by those of ordinary skill in the art that the implementations described herein may be practiced without these specific details. In other instances, well-known methods, procedures and components have not been described in detail so as not to obscure the implementations described herein. Also, the description is not to be considered as limiting the scope of the implementations described herein.

The detailed description set forth herein in connection with the appended drawings is intended as a description of exemplary an embodiment in which the presently disclosed apparatus and system can be practiced. The term “exemplary” used throughout this description means “serving as an example, instance, or illustration,” and should not necessarily be construed as preferred or advantageous over other an embodiment.

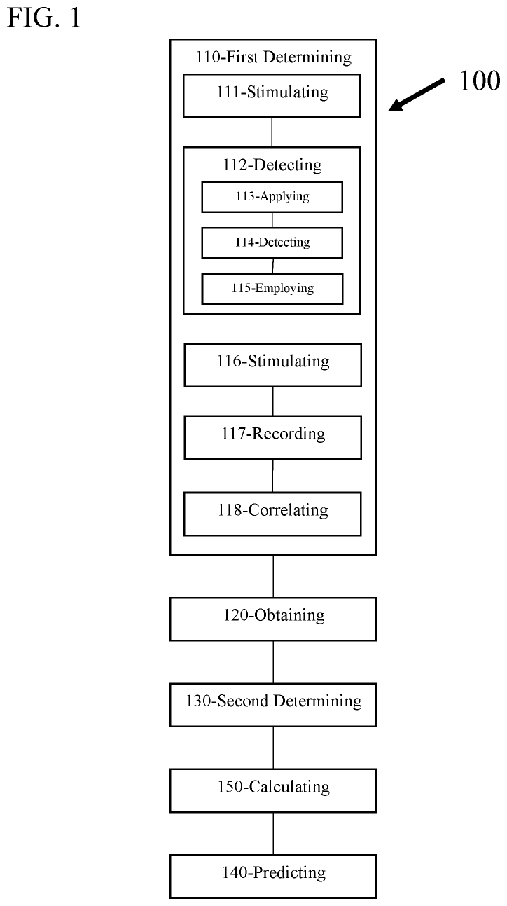

Illustrated in FIG. 1 is a simplified flow diagram of a method 100 of a method for predicting cannabis preferences of a subject. The method may comprise first determining 110 phenotypic expression of T2Rs and/or T1Rs, wherein the determining may be from an actual test administered to detect the functionality of T2Rs and/or T1Rs. The method may further comprise obtaining 120 taste preference information from the subject. The method may further comprise second determining 130 the subject's taste preference scores for one or more cannabis administered to the subject. The test method may further comprise predicting 140 cannabis preferences for the subject.

Referring to FIG. 1, method 100 may further include calculating 150 cannabis bin scores based on the first determining phenotypic expression, the obtaining taste preferences information, and the second determining the subject's tast preference scores. The cannabis bins may comprise one or more cannabis bins selected from the group consisting of a cannabis bin differentiated by taste. The cannabis bins may comprise one or more cannabis bins selected from the group consisting of a first cannabis bin for bitter and/or sweet cannabis, a second cannabis bin consisting of bland choices both away from sweet and bitter, and a third cannabis bin consisting of a broad range of cannabis choices.

Referring to FIG. 1, method 100 may further include the cannabis bin scores used to predict that the subject will prefer cannabis from a cannabis bin having a higher cannabis bin score than a cannabis bin having a lower cannabis bin score.

Referring to FIG. 1, the method 100 may further include the first determining 110 may comprise stimulating 111 T2Rs and/or T1Rs of a human subject with one or more agonists, and detecting 112 one or more products released as a result of stimulation of the T2Rs and/or T1Rs, wherein the detecting of the products comprises a method comprising either
A-i) applying 113 one or more reagents which interact with one or more of the products released because of the stimulation of the T2Rs and/or T1Rs, and
A-ii) detecting 114 the interaction of the reagent with one or more products, optionally by employing chemiluminescence, an electrochemical sensor, or an optical sensor, to detect the interaction of one or more reagents with one or more of the products, or
B) employing 115 chemiluminescence, an electrochemical sensor, or an optical sensor to detect one or more of the products released because of the stimulation of the T2Rs and/or T1Rs.

Referring to FIG. 1, the agonists may be separately applied 111 prior to the reagent 113 which interacts with one or more of the products as a result of stimulation of T2Rs and/or T1Rs.

Referring to FIG. 1, the agonist and the reagent that interacts with one or more products released as a result of stimulation of T2Rs and/or T1Rs may be contained on or the same test medium.

Referring to FIG. 1, the agonists may be selected from the group consisting of caffeine, denatonium, strychnine, quinine, terpenes, phenylthiocarbamide, thiourea, sodium benzoate, and any two or more of the foregoing.

Referring to FIG. 1, the detecting of the products may comprise applying a test medium containing the reagents which interact with the products.

Referring to FIG. 1, the reagents which interact with the products released as a result of stimulation of T2Rs and/or T1Rs may comprise a Griess reagent.

Referring to FIG. 1, the agonist(s) may be selected from caffeine, denatonium, strychnine, quinine, terpenes, phenylthiocarbamide, thiourea, sodium benzoate, and any two or more of the foregoing, and wherein the detecting of the products may comprise applying a test medium containing the reagents which interact with the products. The reagents which interact with the products released as a result of stimulation of T2Rs and/or T1Rs may comprise a Griess reagent.

Referring to FIG. 1, the first determining may further comprise
i) stimulating 116 T2Rs and/or T1Rs by exposing at least a portion of tissue, for example, the mouth of the human subject to one or more agonists,
ii) recording 117 a discerned level of taste perception by the human subject after the stimulation, and
iii) correlating 118 the discerned level of taste perception to the level of phenotypic expression of the T2Rs and/or T1Rs.

Referring to FIG. 1, the agonists for stimulating 116 may be selected from the group consisting of caffeine, denatonium, strychnine, quinine, terpenes, phenylthiocarbamide, thiourea, sodium benzoate, and any two or more of the foregoing.

Referring to FIG. 1, the agonists for stimulating 116 may be selected from the group consisting of caffeine, denatonium, strychnine, quinine, xylitol, grapefruit seed extract or naringenin, a terpene, and any two or more of the foregoing.

Referring to FIG. 1 steps 116 and 117 may be repeated one or more times, and the stimulating 116 by each of one or more different agonists may be sequential, the recording 117 of each discerned level of taste perception by the human subject may occur after each stimulation, and the correlating 118 may be of one or more of the discerned levels of taste perception to the level of phenotypic expression of the T2Rs and/or T1Rs.

Referring to FIG. 1, the agonists for stimulating 116 may be selected from the group consisting of caffeine, denatonium, strychnine, quinine, terpenes, phenylthiocarbamide, thiourea, sodium benzoate, and any two or more of the foregoing.

Referring to FIG. 1, the method may be performed by providing a computing platform that enables the execution of the method, for recommending cannabis by determining taste perception of an individual and determining cannabis preferences in relation to taste perception.

Referring to FIG. 1, the method may further comprise formulating and making cannabis in relation to determining taste perception of an individual by diagnostic tests to establish taster group and determining cannabis preferences in relation to taste perception

Referring to FIG. 1, the method may comprise conducting cannabis tasting events including use of diagnostic test kits to determine taste perception of an individual and determine cannabis preferences in relation to taste perception for recommending cannabis in relation to such taste perception.

Referring to FIG. 1, the method may comprise advising cannabis makers and suppliers in relation to taste perception of consumers, determined in relation to diagnostic tests establishing taster group and preferences in relation to taste perception.

Referring to FIG. 1, the method may comprise using a test kit configured to enable performing said method, said test kit comprising at least one of said agonists contained in at least one test medium for stimulating 116.

Referring to FIG. 1, the method may comprise using a test kit configured to enable performing said method, said test kit comprising at least one of said agonists to stimulate 111, contained in at least one test medium, a reagent in a test medium to apply 113, said reagent capable of interaction with a product released in relation to said stimulating, said interaction providing a detectable phenomenon.

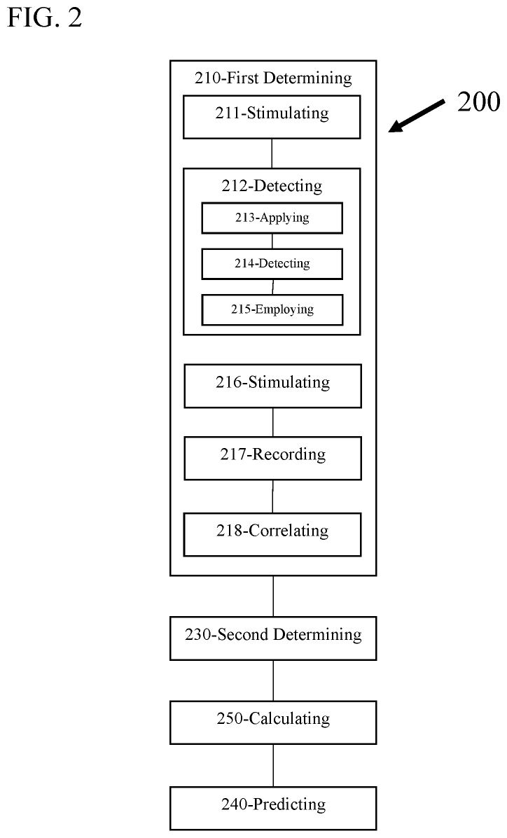

Illustrated in FIG. 2 is a simplified flow diagram of a method 200 of a method for predicting cannabis preferences of a subject. The method may comprise first determining 210 phenotypic expression of T2Rs and/or T1Rs, wherein the determining may be from an actual test administered to detect the functionality of T2Rs and/or T1Rs. The method may also include a second determining 230 the subject's taste preference scores for one or more cannabis administered to the subject. The method may also include predicting 240 cannabis preferences for the subject. The method may also further comprise calculating 250 cannabis bin scores based on the first determining phenotypic expression, and the second determining the subject's tast preference scores.

Referring to FIG. 2, the method may comprise the cannabis bins comprising one or more cannabis bins selected from the group consisting of a first cannabis bin differentiated by taste.

Referring to FIG. 2, the method may comprise the cannabis bins comprising one or more cannabis bins selected from the group consisting of a first cannabis bin for bitter and/or sweet cannabis, a second cannabis bin consisting of bland choices both away from sweet and bitter, and a third cannabis bin consisting of a broad range of cannabis choices.

Referring to FIG. 2, the method may comprise the cannabis bin scores being used to predict that the subject will prefer cannabis from a cannabis bin having a higher cannabis bin score than a cannabis bin having a lower cannabis bin score.

Referring to FIG. 2, the method may comprise the first determining 210 further comprising stimulating 211 T2Rs and/or T1Rs of a human subject with one or more agonists, and detecting 212 one or more products released as a result of stimulation of the T2Rs and/or T1Rs, wherein the detecting of the products comprises a method comprising either
A-i) applying 213 one or more reagents which interact with one or more of the products released because of the stimulation of the T2Rs and/or T1Rs, and
A-ii) detecting 214 the interaction of the reagent with one or more products, optionally by employing chemiluminescence, an electrochemical sensor, or an optical sensor, to detect the interaction of one or more reagents with one or more of the products, or
B) employing 215 chemiluminescence, an electrochemical sensor, or an optical sensor to detect one or more of the products released because of the stimulation of the T2Rs and/or T1Rs.

Referring to FIG. 2, the method may further comprise the agonists being separately applied prior to the reagent which interacts with one or more of the products as a result of stimulation of T2Rs and/or T1Rs.

Referring to FIG. 2, the method may comprise the agonist and the reagent that interacts with one or more products released as a result of stimulation of T2Rs and/or T1Rs may be contained on or the same test medium.

Referring to FIG. 2, the method may further comprise the agonists being selected from the group consisting of caffeine, denatonium, strychnine, quinine, terpenes, phenylthiocarbamide, thiourea, sodium benzoate, and any two or more of the foregoing.

Referring to FIG. 2, the method may further comprise the detecting of the products comprises applying a test medium containing the reagents which interact with the products.

Referring to FIG. 2, the method may further comprise the reagents which interact with the products released as a result of stimulation of T2Rs and/or T1Rs comprise a Griess reagent.

Referring to FIG. 2, the method may further comprise the agonist(s) may be selected from caffeine, denatonium, strychnine, quinine, terpenes, phenylthiocarbamide, thiourea, sodium benzoate, and any two or more of the foregoing, and wherein the detecting of the products comprises applying a test medium containing the reagents which interact with the products.

Referring to FIG. 2, the method may further comprise the reagents which interact with the products released as a result of stimulation of T2Rs and/or T1Rs comprise a Griess reagent.

Referring to FIG. 2, the method may further comprise the first determining 210 further comprises
i) stimulating 216 T2Rs and/or T1Rs by exposing at least a portion of tissue, for example, the mouth of the human subject to one or more agonists,
ii) recording 217 a discerned level of taste perception by the human subject after the stimulation, and
iii) correlating 218 the discerned level of taste perception to the level of phenotypic expression of the T2Rs and/or T1Rs.

Referring to FIG. 2, the method for stimulating 216 may further include the agonists may be selected from the group consisting of caffeine, denatonium, strychnine, quinine, terpenes, phenylthiocarbamide, thiourea, sodium benzoate, and any two or more of the foregoing.

Referring to FIG. 2, the method may further include first determining 210 further comprises wherein the stimulating 216 may comprise agonist being selected from the group consisting of caffeine, denatonium, strychnine, quinine, xylitol, grapefruit seed extract or naringenin, a terpene, and any two or more of the foregoing.

Referring to FIG. 2, the method may further include steps 216 and 217 may be repeated one or more times, and the stimulating 216 by each of one or more different agonists may be sequential, the recording 217 of each discerned level of taste perception by the human subject occurs after each stimulation, and the correlating 218 may be of one or more of the discerned levels of taste perception to the level of phenotypic expression of the T2Rs and/or T1Rs.

Referring to FIG. 2, the method may further include the stimulating 216 with at least one agonist which may be selected from the group consisting of caffeine, denatonium, strychnine, quinine, terpenes, phenylthiocarbamide, thiourea, sodium benzoate, and any two or more of the foregoing.

Referring to FIG. 2, the method may further include providing a computing platform that enables the execution of the method, for recommending cannabis by determining taste perception of an individual and determining cannabis preferences in relation to taste perception.

Referring to FIG. 2, the method may further include formulating and making cannabis in relation to determining taste perception of an individual by diagnostic tests to establish taster group and determining cannabis preferences in relation to taste perception

Referring to FIG. 2, the method may further include conducting cannabis tasting events including use of diagnostic test kits to determine taste perception of an individual and determine cannabis preferences in relation to taste perception for recommending cannabis in relation to such taste perception.

Referring to FIG. 2, the method may further include advising cannabis makers and suppliers in relation to taste perception of consumers, determined in relation to diagnostic tests establishing taster group and preferences in relation to taste perception.

Referring to FIG. 2, the method may further include using a test kit configured to enable performing said method, said test kit comprising at least one of said agonists contained in at least one test medium.

Referring to FIG. 2, the method may further include a test kit configured to enable performing said method, said test kit comprising at least one of said agonists contained in at least one test medium. The test include may further include a reagent in a test medium, said reagent capable of interaction with a product released in relation to said stimulating, said interaction providing a detectable phenomenon.

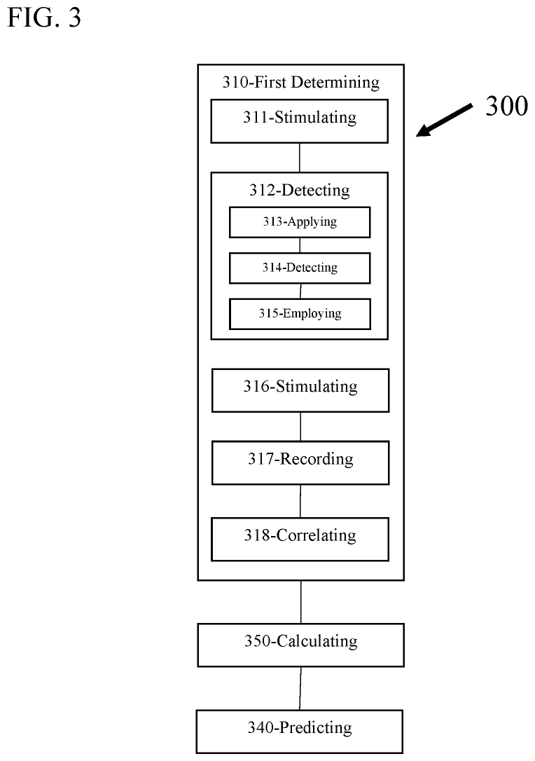

Illustrated in FIG. 3 is a simplified flow diagram of a method 300 of a method for predicting cannabis preferences of a subject, the method comprising first determining 310 phenotypic expression of T2Rs and/or T1Rs, wherein the determining may be from an actual test administered to detect the functionality of T2Rs and/or T1Rs; predicting 340 cannabis preferences for the subject.

Referring to FIG. 3, the method may further include calculating 350 cannabis bin scores based on the first determining phenotypic expression.

Referring to FIG. 3, the method may further include cannabis bins that may comprise one or more cannabis bins selected from the group consisting of.

Referring to FIG. 3, the method may further include cannabis bins that may comprise one or more cannabis bins selected from the group consisting of a first cannabis bin for bitter and/or sweet cannabis, a second cannabis bin consisting of bland choices both away from sweet and bitter, and a third cannabis bin consisting of a broad range of cannabis choices.

Referring to FIG. 3, the method may further include cannabis bin scores that may be used to predict that the subject will prefer cannabis from a cannabis bin having a higher cannabis bin score than a cannabis bin having a lower cannabis bin score.

Referring to FIG. 3, the method may further include the first determining 310 further comprising stimulating 311 T2Rs and/or T1Rs of a human subject with one or more agonists, and detecting 312 one or more products released as a result of stimulation of the T2Rs and/or T1Rs, wherein the detecting of the products comprises a method comprising either
A-i) applying 313 one or more reagents which interact with one or more of the products released because of the stimulation of the T2Rs and/or T1Rs, and
A-ii) detecting 314 the interaction of the reagent with one or more products, optionally by employing chemiluminescence, an electrochemical sensor, or an optical sensor, to detect the interaction of one or more reagents with one or more of the products, or
B) employing 315 chemiluminescence, an electrochemical sensor, or an optical sensor to detect one or more of the products released because of the stimulation of the T2Rs and/or T1Rs and/or T1Rs.

Referring to FIG. 3, the method may further comprise the agonists may be separately applied prior to the reagent which interacts with one or more of the products as a result of stimulation of T2Rs and/or T1Rs and/or T1Rs.

Referring to FIG. 3, the method may further comprise the agonist and the reagent that interacts with one or more products released as a result of stimulation of T2Rs and/or T1Rs and/or T1Rs may be contained on or the same test medium.

Referring to FIG. 3, the method may further comprise the agonists being selected from the group consisting of caffeine, denatonium, strychnine, quinine, terpenes, phenylthiocarbamide, thiourea, sodium benzoate, and any two or more of the foregoing.

Referring to FIG. 3, the method may further comprise the detecting 312 of the products comprises applying a test medium containing the reagents which interact with the products.

Referring to FIG. 3, the method may further comprise the reagents which interact with the products released as a result of stimulation of T2Rs and/or T1Rs and/or T1Rs comprise a Griess reagent.

Referring to FIG. 3, the method may further the agonist(s) being selected from caffeine, denatonium, strychnine, quinine, terpenes, phenylthiocarbamide, thiourea, sodium benzoate, and any two or more of the foregoing, and wherein the detecting of the products comprises applying a test medium containing the reagents which interact with the products.

Referring to FIG. 3, the test method may further comprise the reagents which interact with the products released as a result of stimulation of T2Rs and/or T1Rs comprise a Griess reagent.

Referring to FIG. 3, the method may further comprise the first determining 310 further comprising:
i) stimulating 316 T2Rs and/or T1Rs and/or T1Rs by exposing at least a portion of tissue, for example, the mouth of the human subject to one or more agonists,
ii) recording 317 a discerned level of taste perception by the human subject after the stimulation, and
iii) correlating 318 the discerned level of taste perception to the level of phenotypic expression of the T2Rs and/or T1Rs and/or T1Rs.

Referring to FIG. 3, the method may further comprise the agonists being selected from the group consisting of caffeine, denatonium, strychnine, quinine, terpenes, phenylthiocarbamide, thiourea, sodium benzoate, and any two or more of the foregoing.

Referring to FIG. 3, the method may further comprise the first determining 310 further comprises wherein the agonist may be selected from the group consisting of caffeine, denatonium, strychnine, quinine, xylitol, grapefruit seed extract or naringenin, a terpene, and any two or more of the foregoing.

Referring to FIG. 3, the method may further comprise steps 316 and 317 may be repeated one or more times, and the stimulating 316 by each of one or more different agonists may be sequential, the recording 317 of each discerned level of taste perception by the human subject occurs after each stimulation, and the correlating 318 may be of one or more of the discerned levels of taste perception to the level of phenotypic expression of the T2Rs and/or T1Rs and/or T1Rs.

Referring to FIG. 3, the method may further comprise the agonists may be selected from the group consisting of caffeine, denatonium, strychnine, quinine, terpenes, phenylthiocarbamide, thiourea, sodium benzoate, and any two or more of the foregoing.

Referring to FIG. 3, the method may further include providing a computing platform that enables the execution of the method, for recommending cannabis by determining taste perception of an individual and determining cannabis preferences in relation to taste perception.

Referring to FIG. 3, the method may further comprise formulating and making cannabis in relation to determining taste perception of an individual by diagnostic tests to establish taster group and determining cannabis preferences in relation to taste perception.

Referring to FIG. 3, the method may further comprise conducting cannabis tasting events including use of diagnostic test kits to determine taste perception of an individual and determine cannabis preferences in relation to taste perception for recommending cannabis in relation to such taste perception.

Referring to FIG. 3, the method may further comprise advising cannabis makers and suppliers in relation to taste perception of consumers, determined in relation to diagnostic tests establishing taster group and preferences in relation to taste perception.

Referring to FIG. 3, the method may further comprise using a test kit configured to enable performing said method, said test kit comprising: at least one of said agonists contained in at least one test medium.

Referring to FIG. 3, the method may further comprise using a test kit configured to enable performing said method, said test kit comprising: at least one of said agonists contained in at least one test medium. The test kit may further comprise a reagent in a test medium, said reagent capable of interaction with a product released in relation to said stimulating, said interaction providing a detectable phenomenon.

What is claimed is:

1. A method for predicting cannabis preferences of a subject, the method comprising:
first determining phenotypic expression of T2Rs and/or T1Rs, wherein the determining is from an actual test administered to detect the functionality of T2Rs and/or T1Rs;
obtaining taste preference information from the subject;
second determining the subject's taste preference scores for one or more cannabis administered to the subject;
predicting cannabis preferences for the subject.

2. The method of claim 1, further comprising: calculating cannabis bin scores based on the first determining phenotypic expression, the obtaining taste preferences information, and the second determining the subject's tast preference scores.

3. The method of claim 2, wherein the cannabis bins comprise one or more cannabis bins selected from the group consisting of cannabis bins differentiated by taste.

4. The method of claim 2, wherein the cannabis bins comprise one or more cannabis bins selected from the group consisting of a first cannabis bin for bitter and/or sweet cannabis, a second cannabis bin consisting of bland choices both away from sweet and bitter, and a third cannabis bin consisting of a broad range of cannabis choices.

5. The method of claim 2, wherein the cannabis bin scores are used to predict that the subject will prefer cannabis from a cannabis bin having a higher cannabis bin score than a cannabis bin having a lower cannabis bin score.

6. The method of claim 1, wherein the first determining further comprises:
stimulating T2Rs and/or T1Rs of a human subject with one or more agonists, and detecting one or more products released as a result of stimulation of the T2Rs and/or T1Rs, wherein the detecting of the products comprises a method comprising either A-i) applying one or more reagents which interact with one or more of the products released because of the stimulation of the T2Rs and/or T1Rs, and A-ii) detecting the interaction of the reagent with one or more products, optionally by employing chemiluminescence, an electrochemical sensor,
or an optical sensor, to detect the interaction of one or more reagents with one or more of the products, or
B) employing chemiluminescence, an electrochemical sensor, or an optical sensor to detect one or more of the products released because of the stimulation of the T2Rs and/or T1Rs.

7. The test method as in claim 6 wherein the agonists are separately applied prior to the reagent which interacts with one or more of the products as a result of stimulation of T2Rs and/or T1Rs.

8. The test method as in claim 6 wherein the agonist and the reagent that interacts with one or more products released as a result of stimulation of T2Rs and/or T1Rs are contained on or in the same test medium.

9. The test method as in claim 6 wherein the agonists are selected from the group consisting of caffeine, denatonium, strychnine, quinine, terpenes, phenylthiocarbamide, thiourea, sodium benzoate, and any two or more of the foregoing.

10. The test method as in claim 6 wherein the detecting of the products comprises applying a test medium containing the reagents which interact with the products.

11. The test method as in claim 6 wherein the agonist(s) are selected from caffeine, denatonium, strychnine, quinine, terpenes, phenylthiocarbamide, thiourea, sodium benzoate, and any two or more of the foregoing, and wherein the detecting of the products comprises applying a test medium containing the reagents which interact with the products.

12. The test method as in claim 11 wherein the reagents which interact with the products released as a result of stimulation of T2Rs and/or T1Rs comprise a Griess reagent.

13. The method of claim 1, wherein the first determining further comprises:
i) stimulating T2Rs and/or T1Rs by exposing at least a portion of tissue, for example, the mouth of the human subject to one or more agonists,
ii) recording a discerned level of taste perception by the human subject after the stimulation, and
iii) correlating the discerned level of taste perception to the level of phenotypic expression of the T2Rs and/or T1Rs.

14. The method according to claim 13 wherein the agonists are selected from the group consisting of caffeine, denatonium, strychnine, quinine, terpenes, phenylthiocarbamide, thiourea, sodium benzoate, and any two or more of the foregoing.

15. The method according to claim 14 The method of claim 1, wherein the first determining further comprises wherein the agonist is selected from the group consisting of caffeine, denatonium, strychnine, quinine, xylitol, grapefruit seed extract or naringenin, a terpene, and any two or more of the foregoing.

16. The method according to claim 14 wherein:
steps i) and ii) are repeated one or more times, and the stimulating by each of one or more different agonists is sequential,
the recording of each discerned level of taste perception by the human subject occurs after each stimulation, and
the correlating is of one or more of the discerned levels of taste perception to the level of phenotypic expression of the T2Rs and/or T1Rs.

17. The method according to claim 14 wherein the agonists are selected from the group consisting of caffeine, denatonium, strychnine, quinine, terpenes, phenylthiocarbamide, thiourea, sodium benzoate, and any two or more of the foregoing.

18. A method for predicting cannabis preferences of a subject, the method comprising:
first determining phenotypic expression of T2Rs and/or T1Rs, wherein the determining is from an actual test administered to detect the functionality of T2Rs and/or T1Rs;
second determining the subject's taste preference scores for one or more cannabis administered to the subject;
predicting cannabis preferences for the subject.

19. The method of claim 18, further comprising:
calculating cannabis bin scores based on the first determining phenotypic expression, and the second determining the subject's taste preference scores.

20. The method of claim 19, wherein the cannabis bins comprise one or more cannabis bins selected from the group consisting of cannabis bins differentiated by taste.

21. The method of claim 20, wherein the cannabis bins comprise one or more cannabis bins selected from the group consisting of a first cannabis bin for bitter and/or sweet cannabis, a second cannabis bin consisting of bland choices both away from sweet and bitter, and a third cannabis bin consisting of a broad range of cannabis choices.
